(19) United States
(12) Patent Application Publication
(10) Pub. No.: US 2021/0338586 A1
(43) Pub. Date: Nov. 4, 2021

(54) STABLE LYOPHILIZED DOSAGE FORM OF PROTEIN

(71) Applicant: LUPIN LIMITED, Mumbai (IN)

(72) Inventors: Prasanna DEVARANENI, Pune (IN); Vaibhav DEOKAR, Pune (IN); Rustom MODY, Pune (IN)

(73) Assignee: LUPIN LIMITED, Mumbai (IN)

(21) Appl. No.: 17/255,532

(22) PCT Filed: Jun. 26, 2019
(86) PCT No.: PCT/IB2019/055370

§ 371 Date: Dec. 23, 2020

(30) Foreign Application Priority Data
Jun. 26, 2018 (IN) 201821023751

(51) Int. Cl. A61K 9/19; A61K 47/18; A61K 38/17; A61K 47/26; A61K 9/08

(52) U.S. Cl. CPC A61K 9/19; A61K 47/183; A61K 9/08; A61K 47/186; A61K 47/26; A61K 38/1793

(57) ABSTRACT

A stable lyophilized dosage form of protein is provided that comprises a lyophilate and a reconstitution liquid such that upon reconstitution the formulation will be qualitatively and quantitatively same as that of an already established or developed ready-to-use liquid dosage form of the same protein.

### FIELD OF INVENTION

The present invention relates to a stable lyophilized dosage form of protein pharmaceuticals. Specifically the present invention relates to a stable lyophilized dosage form of protein which upon reconstitution has the same composition as that of an already established or developed ready-to-use liquid dosage form of the same protein.

### BACKGROUND OF INVENTION

Advances in biotechnology have made it possible to produce a variety of proteins for pharmaceutical applications using recombinant DNA techniques. Because proteins are larger and more complex than traditional organic and inorganic drugs (i.e. possessing multiple functional groups in addition to complex three-dimensional structures), the formulation of such proteins poses special problems.

Commercially biopharmaceuticals are available in various dosage forms. Identifying means to convert an already established or developed dosage form into another dosage form would be advantageous in terms of cost and time during development. Further, using the same composition for two different dosage forms can save the time and cost of separate clinical trials for both in some instances.

However different dosage forms have their own attributes, for instance a tablet needs to meet established standards of disintegration time, hardness, dissolution etc. Owing to the different attributes of different dosage forms it is difficult to employ an already established or developed composition of one dosage form into another dosage form.

Particularly for injectable biopharmaceuticals, ready-to-use liquid and lyophilized formulation remain the primary dosage forms. It is difficult to have the same composition for ready-to-use liquid formulation and lyophilized dosage forms due to uncertainty during development, most importantly due to different stability requirements of drug molecule in liquid and in lyophilized formulations.

From a commercial point of view, direct conversion of already established or developed ready-to-use liquid composition of protein into a lyophilized dosage form may pose additional challenges for development due to specific attributes of lyophilized dosage form such as 1) elegant & stable cake, which is free from collapse or melt back 2) minimum reconstitution time 3) minimum particulates upon reconstitution 4) long term stability. Similarly conversion of already established or developed lyophilized dosage form into a ready-to-use liquid formulation, both having the same composition, also has its own challenges.

Surprisingly inventors of the present invention found that a stable lyophilized dosage form of protein which upon re-constitution has the same composition as that of ready-to-use liquid dosage form of the same protein, containing commercially desired attributes, can be prepared if the excipients of ready-to-use liquid formulation are divided in two portions of lyophilized formulation i.e. lyophilate and reconstitution liquid.

### SUMMARY OF THE INVENTION

It is therefore among the objects of the present invention to provide in certain of its preferred aspects and preferred embodiments each and all of the following.

In an embodiment, the invention is related to a stable lyophilized dosage form of protein which upon re-constitution is qualitatively as well as quantitatively same as that of ready-to-use liquid dosage form of the same protein.

In a preferred embodiment the invention relates to a stable lyophilized dosage form of protein which upon re-constitution is qualitatively as well as quantitatively same as that of ready-to-use liquid dosage form of the same protein wherein the lyophilized dosage form comprises of a lyophilate and a reconstitution liquid.

In yet another embodiment of the invention, a stable lyophilized dosage form of protein comprising a lyophilate and a reconstitution liquid, forms a solution upon reconstitution which is qualitatively as well as quantitatively same as that of ready-to-use liquid dosage form of the same protein.

In an embodiment, the invention is related to a stable lyophilized dosage form of a protein comprising a lyophilate with reduced concentration of salt. Surprisingly, a stabilized lyophilized formulation of a protein having an elegant cake can be prepared by limiting the salt concentration from the lyophilate during lyophilization process.

In another embodiment, a stable lyophilized dosage form of protein comprising a lyophilate with a reduced concentration of salt as well as buffer. Preferably elegant cake formation can be prepared by controlling the ionic strength of lyophilate.

In one embodiment of the present invention there is provided a lyophilized dosage form of protein wherein the lyophilate comprises at least a protein and any one or a combination of buffer(s), bulking agent(s) and stabilizer(s) and the reconstitution solution comprises salt(s) and optionally buffer(s), bulking agent(s), solvent(s), preservative(s) or a combination thereof.

In another embodiment, a stable lyophilized dosage form of protein comprises a lyophilate with salt at a concentration of less than or equal to 150 mM and buffer at a concentration of less than or equal to 32 mM respectively.

In yet another embodiment, a stable lyophilized dosage form of protein comprises a lyophilate with salt at a concentration of less than or equal to 150 mM along with buffer at a concentration of less than or equal to 32 mM respectively and a reconstitution solution comprising salt(s) and optionally buffer(s), bulking agent(s), solvent(s), preservative(s) or a combination thereof.

In yet another embodiment, a stable lyophilized composition of protein comprises a lyophilate with salt at a concentration of less than or equal to 150 mM along with buffer at a concentration of less than or equal to 32 mM respectively and a reconstitution solution comprising salt(s) and optionally buffer(s) and bulking agent(s) or a combination thereof, wherein lyophilized dosage form is qualitatively as well as quantitatively same as that of ready-to-use liquid dosage form of the same protein.

In certain embodiments, the invention relates to a stable lyophilized dosage form of a protein comprising lyophilate which is substantially free of salt. “Substantially free” as used herein, refers to a composition or formulation without additional salts. It is to be understood that the buffer(s) itself may comprise some amount of salt(s). The salt component of a composition refers to a salt apart from the salt present in the aqueous buffer.

In yet another embodiment of the present invention there is provided a composition of lyophilized dosage form of protein which is stable at real time and accelerated stress stability conditions for at least six months and at stress stability conditions for at least one month.

In another embodiment, the invention is related to a process for preparation of a stable lyophilized dosage form of protein.

In yet another embodiment, a process for producing a stable lyophilized dosage form of protein comprises:
a) Freezing: involves equilibrating the vials on the shelf at same temperature (˜5° C.) and then freezing the solution at defined rates
b) Annealing: heating the frozen sample containing protein and buffer system, stabilizer and bulking agent below the collapse temperature.
c) Drying the annealed composition under further reduced pressure at a primary drying temperature for a time effective to reduce the moisture content; and
d) Further drying the composition under the further reduced pressure and increased temperature at a second drying temperature for a time effective to further reduce the moisture content of the composition, thereby producing the lyophilized protein composition.

In another embodiment, a process for producing a stable lyophilized dosage form of protein comprises the following steps:
a. Freezing by reducing the temperature of a lyophilate comprising a protein and a bulking agent to a first minimum temperature of −40° C. or less;
b. Annealing by raising the temperature of the composition to an annealing temperature between −25° C. and 0° C.;
c. Holding the composition at the annealing temperature for one hour or more;
d. Reducing the temperature of the composition to a second minimum temperature of −40° C. or less;
e. Raising the temperature of the composition to a first drying temperature of or above −10° C.;
f Holding the composition at the primary drying temperature for five hours or more;
g. Raising the temperature of the composition to a secondary drying temperature;
h. Holding the composition at the secondary drying temperature for two hours or more until the composition is dry, thereby producing the protein lyophilate.

In another embodiment the invention is related to a kit containing the pharmaceutical dosage form of the invention.

In yet another embodiment the invention is related to a kit containing the pharmaceutical dosage form of the invention wherein the kit comprises two separate containers.

In another embodiment the invention is related to a kit comprising a stable lyophilized dosage form of protein wherein a lyophilate with a reduced concentration of salt(s) as well as buffer(s) and a reconstitution solution comprising salt(s) and optionally buffer(s) and bulking agent(s) or a combination thereof is provided, wherein the lyophilized dosage form upon re-constitution is qualitatively as well as quantitatively same as that of ready-to-use liquid dosage form of the same protein.

In a preferred embodiment of the invention the protein is etanercept.

The details of one or more embodiments of the invention set forth below are illustrative only and not intended to limit to the scope of the invention. Other features, objects and advantages of the inventions will be apparent from the description and claims.

### BRIEF DESCRIPTION OF THE FIGURES

FIG. 1: Representative lyophilization cycle used for evaluation of different formulations for cake formation.

FIG. 2: Lyophilization of ready-to-use liquid formulation of etanercept.

FIG. 3 (a to d): Elegancy of cake with different bulking agents.

FIG. 4: Cake appearance for formulations with higher excipient concentration and their characteristics.

FIG. 5: Cake appearance for different formulations and their characteristics.

FIG. 6: Stability of formulations 12, 13, 14, 15 and 16 at 40° C. after 2 weeks.

FIG. 7 (a to c): Stability of formulation 16 at real time stability conditions 2° C. to 8° C. up to 6 months by SE-HPLC. (a) % Monomer Content, (b) % HMW Content, (c) % LMW Content.

FIG. 8 (A to c): Stability of formulation 16 at accelerated conditions 25° C./60% RH up to 6 months by SE-HPLC. a) % Monomer Content, (b) % HMW Content, (c) % LMW Content

FIG. 9 (a to c): Stability of formulation 16 at stress stability conditions 40° C./75% RH up to 1 month by SE-HPLC. (a) % Monomer Content, (b) % HMW Content, (C) % LMW Content.

FIG. 10 (a to c): Stability of formulation 16 at stress conditions 40° C./75% RH up to 1 month by HI-HPLC. (a) % Main form (b) % Misfolded form (C) % Degraded form.

### DETAIL DESCRIPTION OF INVENTION

The term “dosage form” as used herein comprises various pharmaceutically acceptable dosage forms including oral solid as well as liquid dosage forms, such as but not limited to, tablets, soft gelatin capsule, capsules (filled with powders, powders for reconstitution, pellets, beads, mini-tablets, pills, micro-pellets, small tablet units, MUPS, disintegrating tablets, dispersible tablets, granules, sprinkles microspheres and multiparticulates), sachets (filled with powders, pellets, beads, mini-tablets, pills, micropellets, small tablet units, MUPS, disintegrating tablets, dispersible tablets, granules, sprinkles microspheres and multiparticulates) and sprinkles, liquids, liquid dispersions, suspensions, solutions, emulsions, sprays, spot-on) and the like; parenteral dosage forms such as liquids, liquid dispersions, suspensions, solutions, emulsions, (rods, capsules, rings), lyophilized formulation for reconstitution or freeze-dried powder, ready-to-use-liquid and the like; topical dosage forms, such as but not limited to, sprays, solutions, suspensions, ointments, drops, in-situ gel, aerosols, ointments, microspheres, creams, gels, patches, films etc.

As used herein, the phrase “composition” or “compositions” may refer to formulation(s) comprising a protein or a combination of proteins and pharmaceutically acceptable excipient(s) and concentration thereof, prepared such that it is suitable for injection and/or administration into an individual in need thereof. A “composition” may also be referred to as a “pharmaceutical composition” or “formulation” or “pharmaceutical formulation”. In certain embodiments, the compositions provided herein are substantially sterile and do not contain any agents that are unduly toxic or infectious to the recipient.

As used herein ready-to-use liquid formulation/dosage form/composition or already established or developed ready-to-use liquid formulation/dosage form/composition refers to a liquid formulation that is supplied in a form which is ready for administration to the patient and is well known to a person skilled in the art.

Lyophilate and reconstitution liquid or liquid for reconstitution or solution for reconstitution are terms well known to a person skilled in the art.

Reconstituted Solution or solution upon reconstitution as used herein refers to the solution obtained after reconstitution of lyophilate with solution for reconstitution which is ready for administration.

Qualitatively as well as quantitatively same as used herein are terms well known to a person skilled in the art.

“Annealing” as used herein means a heat treatment that alters the microstructure of a material and changes its properties, typically by providing defect free crystals and minimizing internal stresses. Annealing typically involves heating to a temperature at which the material is too hard to deform but is soft enough for internal stresses to ease, and then holding the material at that temperature until it is thoroughly equilibrated.

In the context of the present invention, annealing particularly involves holding the lyophilate at an annealing temperature for a defined period to ensure crystallization (relatively free of crystal defects) of the crystallizable components in the formulation, particularly the bulking agent. Annealing ensures that such components, particularly the bulking agent, will be stable and completely crystallize the bulking agent during freezing stage. Instability in crystallization can result in poor cake formation and poor solubility characteristics. Crystallization during the drying stage often causes loss of protein stability in the lyophilate cake and vial breakage, among other deleterious effects. The target product temperature is generally 2 to 5° C. below the Tc or Tg′ to get an elegant cake and a short drying cycle.

Due to the physico-chemical state of a formulation, the lyophilization process is limited by a maximum allowable product temperature which must not be exceeded during the primary drying phase. This temperature is called the “critical formulation temperature” and depends on the type and total amount of the constituents of the aqueous formulation. The critical formulation temperature can be determined by several methods which are all based on an increase in molecular mobility in the product system. For crystalline systems, the maximally tolerable temperature is the eutectic temperature (Tₑᵤₜ) or collapse temperature (T_{c}), which is commonly determined nowadays by Differential Scanning calorimetry (DSC). Eutectic temperatures are high relative to glass transitions and processing of such formulations is easy. For amorphous systems, however, the critical temperature is represented by the glass transition temperature (T_{g}′) of the maximally freeze concentrated solute.

T_{g}′ and T_{c} do not represent the same experimental parameter: T_{g}′ by DSC is a measure of a heat flow difference between an empty reference pan and a sample pan containing a completely frozen structure. Ice is in close contact with the glassy phase during the entire experiment and is not removed by sublimation (atmospheric pressure). In turn, a Freeze-Dry Microscopy measurement imitates the freeze-drying process by applying controlled, low pressure conditions in a special freeze-drying stage which then allows to sublime ice from the sample. The difference between these two methods becomes obvious in the results obtained for the critical formulation temperature: T_{c} values are many times reported higher (2-5° C.) than their corresponding T_{g}′ values.

“Glass transition temperature” or Tg′ is the temperature below which a substance loses its elastic properties and becomes hard and brittle (solid) and above which it is elastic (generally, near the transition temperature of glass).

“Fill volume” is the volume of dosage form filled in the primary container closure in order to achieve the target deliverable volume. The fill volume accounts to the total volume considering hold up volume and losses during administration. The fluid remaining inside the primary container closure is typically referred to as hold-up volume.

“Deliverable volume” is the volume of dosage form that is declared on the label of the drug product.

“Ionic strength” of a solution is a measure of electrolyte concentration and valency of electrolyte.

Formulated bulk solution is solution containing the required active pharmaceutical ingredient formulated along with excipients or is diluted to a required concentration prior to lyophilization to produce the dosage form.

In addition, as used herein, the term “about” may mean that there can be variation in the concentration of a component of the described compositions that can be to 5%, 10%, 15% or up to and including 20% of the given value.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

Any protein may be used in the dosage forms of the invention. In one embodiment, the dosage form comprises fusion protein(s), peptide(s), peptibody, an antibody(s), or an antigen-binding fragment thereof. Types of peptide(s), peptibody and antibody(s) are defined in WO2007014073 and is incorporated by reference herein.

As used herein, “fusion protein(s) comprises a protein fused to an Fc domain that is an antibody constant region, in which the protein means an amino acid polymer made up of several amino acids formed by peptide bonds. Also, a polypeptide has the same meaning as an oligopeptide having a low molecular weight and a protein having a high molecular weight. Thus, as used herein, “a fusion protein of a protein and an Fc domain” has the same meaning as “a fusion protein of a polypeptide and an Fc domain”.

In an embodiment of the invention, the active pharmaceutical ingredient etanercept is used which is obtained from recombinant DNA technology using CHO cells. The concentration of the etanercept in the composition is 10 mg/mL to 100 mg/ml. In a preferred embodiment of the invention, the concentration of etanercept in the composition is 10 mg/mL to 60 mg/ml. In the most preferred embodiment of the invention, the concentration of etanercept in the composition is 20 mg/mL to 60 mg/ml. “High concentration” as used herein with reference to protein therapeutic compositions means 20 mg/ml or more.

“Etanercept” is a biopharmaceutical that treats autoimmune diseases by interfering with tumor necrosis factor by acting as a TNF-alpha inhibitor. Tumor Necrosis Factor alpha (TNF-alpha) is a member of a group of cytokines that stimulate the acute phase reaction, and thus is a cytokine involved in systemic inflammation. TNF-alpha is able to induce inflammation, induce apoptotic cell death, and to inhibit tumorgenesis and viral replication. Dysregulation of TNF-alpha production has been implicated in a variety of human diseases like autoimmune disease, ankylosing spondylitis, juvenile rheumatoid arthritis, psoriasis, psoriatic arthritis, rheumatoid arthritis, Wegener's disease (granulomatosis), Crohn's disease or inflammatory bowel disease, chronic obstructive pulmonary disease (COPD), Hepatitis C, endometriosis, asthma, cachexia, atopic dermatitis, Alzheimer as well as cancer.

Dosage of the etanercept will depend on the disease, severity of condition, patient's clinical history, and response to the (prior) therapy, and will be adjusted and monitored by a physician. The pharmaceutical composition may be administered parenterally, such as subcutaneously, intramuscularly, intravenously, intraperitoneally, intra-cerebrospinally, intra-articularly, intra-synovially and/or intra-thecally by either bolus injection or continuous infusion.

In an embodiment etanercept may be administered in adult or juvenile subject, wherein the amount may range from about 1-80 mg. The dose may be administered once weekly, twice weekly. Further, the doses may be administered weekly, biweekly, or separated by several weeks e.g. three weeks. The therapeutic dose and duration may vary as per patient response and patient requirement.

In another embodiment, a suitable regimen for juvenile and pediatric patients may involve a dose of 0.4 mg/kg to 5 mg/kg of etanercept, administered one or more times per week. In case of adult rheumatoid arthritis, 25 mg twice weekly or 50 mg once weekly etanercept may be administered.

In case of psoriatic arthritis, 25 mg twice weekly or 50 mg once weekly etanercept may be administered. In case of ankylosing spondylitis, 25 mg twice weekly or 50 mg once weekly etanercept may be administered.

In case of adult plaque psoriasis, the recommended dose of etanercept is 25 mg administered twice weekly or 50 mg administered once weekly. In case of pediatric plaque psoriasis, the recommended dose of etanercept is 0.8 mg/Kg weekly with a maximum of 50 mg dose per week.

In case of polyarticular juvenile idiopathic arthritis, the recommended dose of etanercept is 0.8 mg/Kg weekly with a maximum of 50 mg dose per week.

In case of renal and hepatic impairment no dose adjustment is required.

In other embodiments, the compositions described herein are prepared in a bulk formulation and as such, the components of the composition are adjusted so that it is higher than would be required for administration and diluted appropriately prior to administration.

In certain embodiments, the compositions described herein are administered parenterally, e.g., subcutaneously, intramuscularly, intravenously, intraperitoneal, intracerebrospinal, intraarticular, intrasynovial, and/or intrathecal.

As used herein, the phrase “long-term” storage is understood to mean that the composition can be stored for three months or more, for six months or more, or for one year, or two years, or more. Long term storage is also understood to mean that the active polypeptide of the pharmaceutical composition does not substantially lose its activity as compared to the composition at the beginning of storage. The term “stable” with respect to long-term storage refers to the appearance (elegance, compactness, brittleness) of the cake formed by incorporating the compositions and the process for preparing the composition of the invention. Stability of a composition can be assessed based on purity by SE-HPLC (Size exclusion Chromatography) and HI-HPLC (Hydrophobic interaction chromatography).

In other embodiments, the compositions described herein are presented in a vial, pack or dispenser device which may contain one or more unit dosage forms containing the active ingredient.

In another embodiment the invention is related to a kit or container containing the pharmaceutical composition of the invention.

Other aspects of compositions and the process for preparing the composition in accordance with the invention are described in greater detail below and elsewhere herein.

### A. Reconstitution Procedure(s)

Lyophilized compositions in various aspects and embodiments of the invention may be reconstituted by a variety of methods, including, for instance, the procedures specified by a manufacturer and/or supplier for reconstituting a pharmaceutical protein comprising lyophilate composition for veterinary or human use.

### B. Reconstitution Time

For timing reconstitution, a stop watch should be started as soon as the diluent is started into the vial. After the desired volume of diluent has been introduced, the vial is swirled gently for preferably 10 seconds. Reconstitution should be monitored under controlled conditions with a known intensity of light incident on the sample, against uniform black and uniform white backgrounds.

### C. Salt(s)

In certain embodiments, the lyophilate has reduced concentration of salt.

Reduced concentration of salt means salt at a concentration of less than or equal to 150 mM, more preferably less than or equal to 30 mM.

In certain embodiments, lyophilate is substantially free of salt. “Substantially free” as used herein, refers to a composition or formulation without additional salts. It is to be understood that the buffer(s) itself may comprise some amount of salt(s). The salt component of a composition refers to a salt apart from the salt present in the aqueous buffer.

Salts, used herein, can include without limitation sodium chloride, potassium chloride, magnesium sulphate, calcium chloride, magnesium chloride, sodium nitrate, sodium chromate, magnesium dioxide, potassium nitrate, sodium phosphate, potassium phosphate, calcium phosphate, magnesium phosphate, sodium acetate, potassium acetate, calcium acetate or magnesium acetate.

In preferred embodiment, the salt is sodium chloride (NaCl).

### D. Buffer(s)

Buffer(s) in accordance with this aspect of the invention are compatible with the protein appropriate to the desired end use, provide adequate buffering capacity at concentrations consistent with acceptable osmolality, are inert, stable, and have their maximum buffering capacity at or near the desired pH.

A variety of buffers can be used in the lyophilization solution in accordance with various aspects and preferred embodiments of the invention in this regard. (Some of the same buffers can be used to reconstitute lyophilate, as discussed below.) Preferred buffers in this aspect of the invention include Tris, phosphate, citrate and citrate-phosphate, acetate, succinate to mention just a few.

In certain preferred embodiments, the buffer concentration is less than or equal to 32 mM. In another embodiment the buffer concentration is in the range of 5 to 50 mM. Buffers in accordance with this aspect of the invention are effective to maintain appropriate pH. The exact optimal pH will vary from protein to protein. Accordingly, different buffer systems will be more or less better than one another for different proteins. Generally, however, the preferred buffers are effective for pH in the range of 5 to 8, especially in the range of 5.5 to 7.5.

### E. Bulking Agents

A variety of bulking agents can be used in accordance with various aspects and preferred embodiments of the invention herein disclosed. In particular, bulking agents can be used in this regard to facilitate the formation of uniform lyophilate cakes with desired structure and porosity.

Among bulking agents useful in this regard are mannitol, anhydrous lactose, sucrose, D(+)-α,α-trehalose, dextran 40, povidone (PVP K24), glycine, and hydroxyethyl starch. Among particularly preferred bulking agents in this is glycine.

### F. Lyoprotectants/Stabilizers

Lyoprotectants/Stabilizers are substances that, generally, protect proteins against denaturation as a result of lyophilization. In certain aspects and preferred embodiments of the invention thereof, lyoprotectants are used in the lyophilization process, and the resulting lyophilates and compositions produced thereby comprise the same. Among lyoprotectants in accordance with certain preferred embodiments of the invention is sucrose.

### G. Preservative(s)

The lyophilized formulation according to the present invention may further include a preservative. Examples of the preservative may include benzalkonium chloride, benzethonium, chlorhexidine, phenol, m-cresol, benzyl alcohol, methylparaben, propylparaben, chlorobutanol, o-cresol, p-cresol, chloro-cresol, phenylmercuric nitrate, thimerosal, and benzoic acid, but are not limited thereto. These preservatives may be used either alone or in combinations of two or more thereof.

### H. Excipients

In other embodiments, any one of the compositions described herein can comprise an excipient. The excipient may be sucrose, lactose, glycerol, xylitol, sorbitol, Mannitol, maltose, inositol, trehalose, glucose, bovine serum albumin (BSA), human SA or recombinant HA, dextran, PVA, hydroxypropyl methylcellulose (HPMC), polyethyleneimine, gelatin, polyvinylpyrrolidone (PVP), hydroxyethylcellulose (HEC), polyethylene glycol, ethylene glycol, glycerol, dimethylsulfoxide (DMSO), dimethylformamide (DMF), proline, L-serine, sodium glutamic acid, alanine, glycine, lysine hydrochloride, sarcosine, gamma-aminobutyric acid, Tween-20, Tween-80, SDS, polysorbate, polyoxyethylene copolymer, potassium phosphate, sodium acetate, ammonium sulfate, magnesium sulfate, sodium sulfate, 15 trimethylamine N-oxide, betaine, zinc ions, copper ions, calcium ions, manganese ions, magnesium ions, CHAPS, sucrose monolaurate, 2-0-beta-mannoglycerate or a combination thereof. Other excipients can be used, as aspects of the invention are not limited in this regard.

### I. Lyophilization Methods

Various embodiments of aspects of the invention provide lyophilization methods in which a process for producing a stable lyophilized dosage form of protein comprises:
a) Freezing: involves equilibrating the vials on the shelf at same temperature (˜5° C.) and then freezing the solution at defined rates
b) Annealing: heating the frozen sample containing protein and buffer system, stabilizer and bulking agent below the collapse temperature.
c) Drying the annealed composition under further reduced pressure at a primary drying temperature for a time effective to reduce the moisture content; and
d) Further drying the composition under the further reduced pressure and increased temperature at a second drying temperature for a time effective to further reduce the moisture content of the composition, thereby producing the lyophilized protein composition.

In another embodiment is provided a process for producing a stable lyophilized dosage form of protein comprising following steps:
a) Freezing by reducing the temperature of a lyophilate comprising a protein and a bulking agent to a first minimum temperature of −40° C. or less;
b) Annealing by raising the temperature of the composition to an annealing temperature between −25° C. and 0° C.;
c) Holding the composition at the annealing temperature for one hour or more;
d) Reducing the temperature of the composition to a second minimum temperature of −40° C. or less;
e) Raising the temperature of the composition to a first drying temperature of or above −10° C.;
f) Holding the composition at the primary drying temperature for five hours or more;
g) Raising the temperature of the composition to a secondary drying temperature;
h) Holding the composition at the secondary drying temperature for two hours or more until the composition is dry, thereby producing the protein lyophilate.

### EXAMPLES

Enbrel® is a marketed formulation of etanercept for subcutaneous injection, supplied as ready-to-use liquid formulation in prefilled syringe, or as prefilled syringe in an auto injector, or as a lyophilized product in a glass vial which is reconstituted with solution for reconstitution before injection. The composition of Enbrel® ready-to-use liquid and lyophilized dosage form is as follows:

TABLE 1(a)
Composition of Enbrel ® ready-to-use liquid formulation.
Enbrel ® Liquid formulation

| Excipients | Concentration | |
|---|---|---|
| Etanercept | 50 | mg/mL |
| L-arginine HCl | 25 | mM |
| Di Sodium hydrogen Phosphate, 2H20 | 25 | mM |
| Sodium Dihydrogen Phosphate, 2H2O | | |
| Sodium chloride | 100 | mM |

| | |
|---|---|
| Sucrose | 1% |
| pH | 6.3 ± 0.2 |

TABLE 1(b)
Composition of Enbrel ® lyophilized formulation.
Enbrel ® Lyophilized formulation 25 mg/vial

| Excipients | Concentration | |
|---|---|---|
| Etanercept | 25 | mg/mL |
| Mannitol | 40 | mg |
| Sucrose | 10 | mg |
| Tromethamine | 1.2 | mg |
| Reconstituted with Sterile Bacteriostatic | 1.0 | mL |
| Water for Injection, USP (containing | | |
| 0.9% benzyl alcohol) | | |

| | |
|---|---|
| pH | 7.4 ± 0.3 |

The following examples are illustrative of particular aspects and embodiments of the invention and in no way limit its scope. Many other aspects and embodiments of the invention will be immediately clear to those skilled in the art from the contents of this disclosure, and a full understanding of the invention herein disclosed can be obtained only by careful scrutiny of the present disclosure in all its details as it should be understood by the skilled person fully knowledgeable in the pertinent arts.

### Example 1: Lyophilization of Etanercept Ready-to-Use Liquid Formulation

TABLE 2
Composition of etanercept ready-to-use liquid
formulation (Control DS) and role of excipients

| Name of Ingredient | Concentration | | Role |
|---|---|---|---|
| Etanercept | 50 | mg/mL | Active Pharmaceutical |
| | | | Ingredient (API) |
| Glycine | 5.62 | mg/mL | Stabilizer |
| Trisodium citrate dihydrate | 4.5 | mg/mL | Buffer component |
| Sodium dihydrogen phosphate | 2.6 | mg/mL | Buffer component |
| dihydrate | | | |
| Sucrose | 10 | mg/mL | Stabilizer |
| Sodium chloride | 3.8 | mg/mL | Tonicity modifying agent |

| | | |
|---|---|---|
| Water for Injection | q.s. 1 mL | Solvent |

| | |
|---|---|
| pH | 6.3 ± 0.2 |

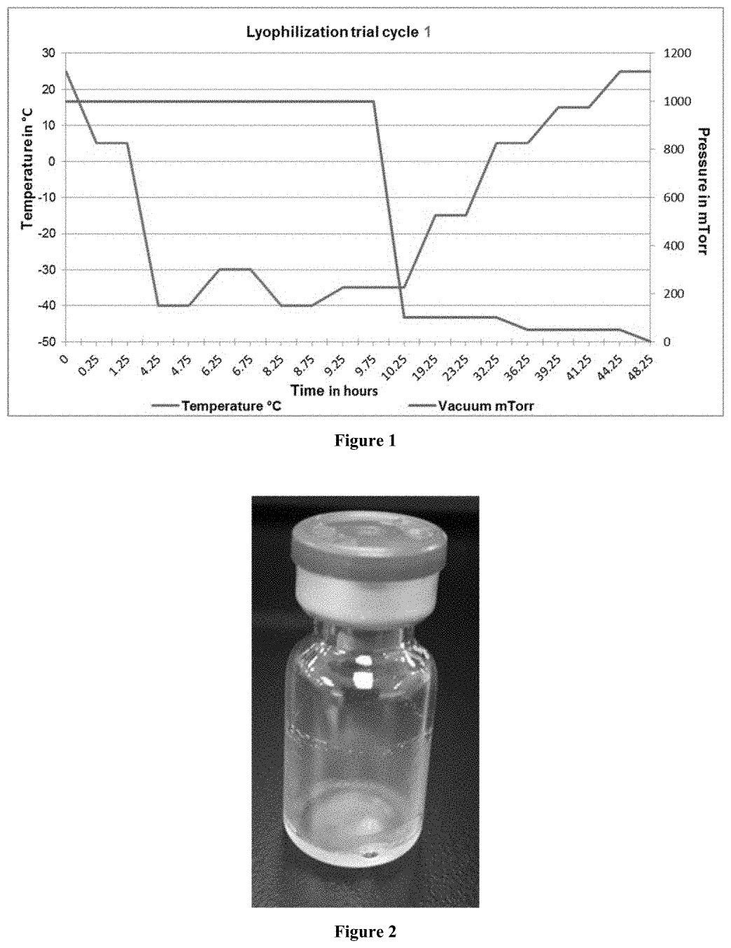

Table 2 enlists the excipients and their role in ready-to-use liquid formulation for etanercept. Glycine and sucrose present in ready-to-use liquid formulation could act as bulking agent and support cake formation. The ready-to-use liquid formulation as such was lyophilized using lyophilisation cycle (FIG. 1) to test the cake formation and its attributes. Surprisingly, no visible cake was observed and the dried material resembled to that of liquid drying (FIG. 2). It is possible that one or more of formulation excipients could be interfering with cake formation. This can happen by excipients modulating physical properties of bulk solution pertaining to freezing and/or drying process. Additionally it could also be possible that cake formation required a critical concentration of bulking agent/s.

### Example 2

### Lyophilization Using Different Bulking Agents

Different bulking agents such as sucrose, trehalose and glycine were evaluated for elegant cake formation using citrate-phosphate and trometamol buffer formulations. The compositions are listed in Table 3.

TABLE 3
Compositions evaluated for cake formation

| Formu- | | Concen- | Mo- | |
|---|---|---|---|---|
| lation | | tration | larity | |
| Number | Excipients | mg/mL | mM | Role of excipient |
| 01 | Glycine | 11.5 | 153.2 | Bulking agent |
| | Tri sodium citrate | 4.5 | 15.3 | Buffering agent |
| | dihydrate | | | |
| | Sodium dihydrogen | 2.6 | 16.7 | Buffering agent |
| | phosphate dihydrate | | | |
| | Sucrose | 10 | 29.2 | Structure stabilizer |
| 02 | Glycine | 23 | 306.4 | Bulking agent |
| | Sucrose | 10 | 29.2 | Structure stabilizer |
| | Trometamol (Tris) | 1.2 | 9.9 | Buffering agent pH 7.4 |
| 03 | Sucrose | 92 | 270.6 | Bulking agent and |
| | | | | Structure stabilizer |
| | Trometamol (Tris) | 1.2 | 9.9 | Buffering agent pH 7.4 |
| 04 | Trehalose | 82 | 216.7 | Bulking agent |
| | Sucrose | 10 | 29.2 | Structure stabilizer |
| | Trometamol (Tris) | 1.2 | 9.9 | Buffering agent pH 7.4 |
| 05 | Glycine | 0.74 | 9.9 | Stabilizer |
| | Tri sodium citrate | 4.5 | 15.3 | Buffering agent pH 6.3 |
| | dihydrate | | | |
| | Sodium dihydrogen | 2.6 | 16.7 | Buffering agent pH 6.3 |
| | phosphate dihydrate | | | |
| | Sodium chloride | 0.29 | 5.0 | Tonicity modifying |
| | | | | agent |
| | Sucrose | 60 | 175.3 | Bulking agent |
| 06 | Glycine | 11.24 | 149.7 | Bulking agent |
| | Tri sodium citrate | 4.5 | 15.3 | Buffering agent pH 6.3 |
| | dihydrate | | | |
| | Sodium dihydrogen | 2.6 | 16.7 | Buffering agent pH 6.3 |
| | phosphate dihydrate | | | |
| | Sucrose | 3 | 8.8 | Structure stabilizer |
| 07 | Glycine | 22.48 | 299.5 | Bulking agent |
| | Tri sodium citrate | 4.5 | 15.3 | Buffering agent pH 6.3 |
| | dihydrate | | | |
| | Sodium dihydrogen | 2.6 | 16.7 | Buffering agent pH 6.3 |
| | phosphate dihydrate | | | |
| | Sucrose | 1 | 29.2 | Structure stabilizer |

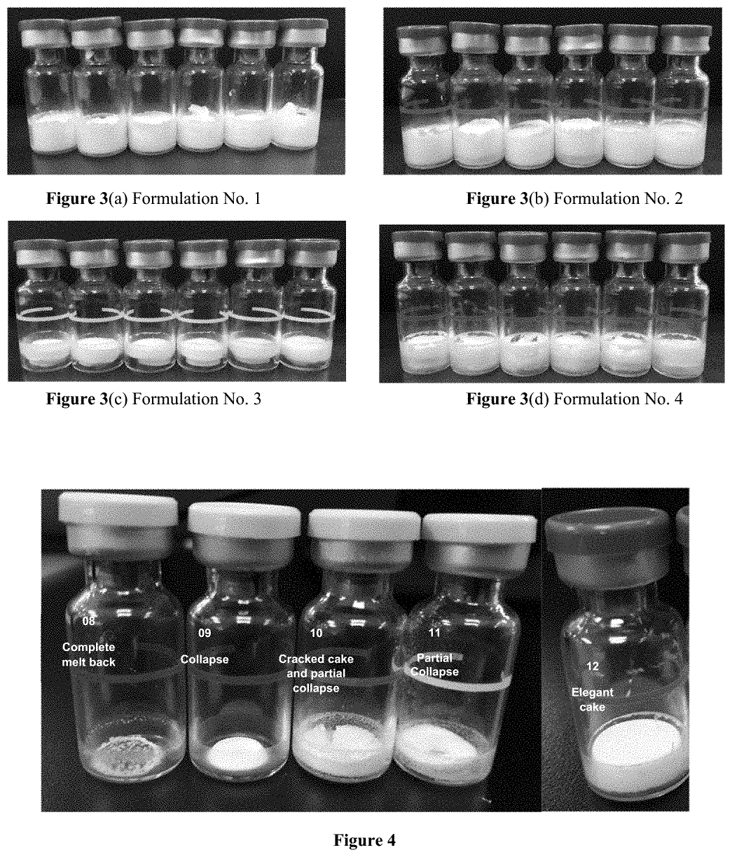

Bulking agents affected the elegancy of cake in both buffer systems (FIG. 3). Glycine as a bulking agent resulted in lustrous cake formation with cake volume equivalent to fill volume. On the other hand partial collapse of cake was observed when trehalose and sucrose were used as bulking agent. However, both, sucrose and trehalose have been historically used as bulking agents, and it is possible that by modifying the lyophilization cycle, a cake with desired attributes can be obtained.

### Example 3: Use of Higher Concentration of Excipients to Obtain Cake Upon Lyophilization

In the example 1, ready-to-use liquid formulation consisted of 50 mg/ml etanercept and the fill volume was 1.0 ml. In this example, etanercept 50 mg/ml with two-fold (2×) concentration of bulking agents and etanercept 75 mg/ml with three-fold (3×) bulking agent concentration were lyophilized. In order to keep the dosage constant (25 mg/vial) fill volumes and reconstitution volumes varied accordingly (Table 4 describes various compositions and their respective reconstitution buffers). In addition, NaCl was excluded along with reduction of buffer strength from concentrated formulations as presence of higher salt content reduces the Tg′ and results in collapse of the cake. The results of the formulation of Table 4 is shown in FIG. 4. Unlike liquid formulation (example 1) where no visible cake was present, concentrating the amount of excipients resulted in cakes with distinct morphological and physical characteristics such as cake volume, compactness, collapsed cake etc. (FIG. 4). For instance, increasing the glycine resulted in collapsed cake while elimination of glycine formed relatively stable cake. However cake became more compact as the fill volume reduced due to increase in concentration of excipients and products. A compact cake is undesirable due to longer reconstitution time required. In addition compact cakes were observed to be brittle and cracking. It is further possible that with suitable modification of lyophilization method, a morphologically suitable cake may be obtained in some of above formulations, if not all. This suggest that concentration of bulking agents is critical for cake formation and their effect is dependent on other excipients. The strategy of using concentrated liquid formulation for lyophilization requires that fill volume for lyophilate and reconstitution volume would be different.

TABLE 4
Use of higher concentration of excipients
to obtain cake upon lyophilization
(A) Formulation 08

| | | Concen- | Mo- | |
|---|---|---|---|---|
| Serial | | tration | larity | |
| Number | Excipients | mg/mL | in mM | Role of excipient |
| 08 | Glycine (3X) | 16.86 | 224.6 | Partial Bulking |
| | | | | agent |
| | Tri sodium citrate | 1.50 | 5.1 | Buffering agent |
| | dihydrate | | | |
| | Sodium dihydrogen | 0.87 | 5.6 | Buffering agent |
| | phosphate dihydrate | | | |
| | Sodium chloride | NIL | NIL | NIL |
| | Sucrose (3X) | 30 | 87.6 | Partial Bulking |
| | | | | agent and structure |
| | | | | stabilizer |

| | |
|---|---|
| Fill Volume in mL | 0.333 |
| DS Concentration | 75 mg/mL |
| mg/mL | |

(B) Reconstitution buffer 08

| Excipient composition Reconstitution | Concentration |
|---|---|
| buffer 08 | in mg/mL |
| Glycine | NIL |
| Tri sodium citrate dihydrate | 4 |
| Sodium dihydrogen phosphate dihydrate | 2.30 |
| Sodium chloride | 3.8 |
| Sucrose | NIL |
| Reconstitution Volume | 1.00 mL |

(C) Formulation 09

| | | Concen- | Mo- | |
|---|---|---|---|---|
| Serial | | tration | larity | |
| Number | Excipients | mg/mL | in mM | Role of excipient |
| 09 | Glycine | NIL | NIL | NIL |
| | Tri sodium citrate | 1.50 | 5.1 | Buffering agent |
| | dihydrate | | | |
| | Sodium dihydrogen | 0.87 | 5.6 | Buffering agent |
| | phosphate dihydrate | | | |
| | Sodium chloride | NIL | NIL | NIL |
| | Sucrose (3X) | 30.00 | 87.6 | Bulking Agent and |
| | | | | structure stabilizer |

| | |
|---|---|
| Fill Volume in mL | 0.333 |
| DS Concentration | 75 mg/mL |
| mg/mL | |

(D) Reconstitution buffer 09

| Excipient composition Reconstitution | Concentration |
|---|---|
| buffer 09 | in mg/mL |
| Glycine | 5.62 |
| Tri sodium citrate dihydrate | 4 |
| Sodium dihydrogen phosphate dihydrate | 2.30 |
| Sodium chloride | 3.8 |
| Sucrose | NIL |
| Reconstitution Volume | 1.00 mL |

(E) Formulation 10

| | | Concen- | Mo- | |
|---|---|---|---|---|
| Serial | | tration | larity | |
| Number | Excipients | mg/mL | in mM | Role of excipient |
| 10 | Glycine (3X) | 16.860 | 224.6 | Bulking Agent |
| | Tri sodium citrate | 1.500 | 5.1 | Buffering agent |
| | dihydrate | | | |
| | Sodium dihydrogen | 0.867 | 5.6 | Buffering agent |
| | phosphate dihydrate | | | |
| | Sodium chloride | NIL | NIL | NIL |
| | Sucrose | NIL | NIL | NIL |

| | |
|---|---|
| Fill Volume in mL | 0.333 |
| DS Concentration | 75 mg/mL |
| mg/mL | |

(F) Reconstitution buffer 10

| Excipient composition Reconstitution | Concentration |
|---|---|
| buffer 10 | in mg/mL |
| Glycine | NIL |
| Tri sodium citrate dihydrate | 4 |
| Sodium dihydrogen phosphate dihydrate | 2.30 |
| Sodium chloride | 3.8 |
| Sucrose | 10 |
| Reconstitution Volume | 1.00 mL |

(G)Formulation 11

| | | Concen- | Mo- | |
|---|---|---|---|---|
| Serial | | tration | larity | |
| Number | Excipients | mg/mL | in mM | Role of excipient |
| 11 | Glycine (3X) | 16.86 | 224.6 | Bulking Agent |
| | Tri sodium citrate | 1.50 | 5.1 | Buffering agent |
| | dihydrate | | | |
| | Sodium dihydrogen | 0.87 | 5.6 | Buffering agent |
| | phosphate dihydrate | | | |
| | Sodium chloride | NIL | NIL | NIL |
| | Sucrose | 10.00 | 29.2 | Structure stabilizer |

| | |
|---|---|
| Fill Volume in mL | 0.333 |
| DS Concentration | 75 mg/mL |
| mg/mL | |

(H)Reconstitution buffer 11

| Excipient composition Reconstitution | Concentration |
|---|---|
| buffer 11 | in mg/mL |
| Glycine | NIL |
| Tri sodium citrate dihydrate | 4 |
| Sodium dihydrogen phosphate dihydrate | 2.30 |
| Sodium chloride | 3.8 |
| Sucrose | NIL |
| Reconstitution Volume | 1.00 mL |

(I) Formulation 12

| | | Concen- | Mo- | |
|---|---|---|---|---|
| Serial | | tration | larity | |
| Number | Excipients | mg/mL | in mM | Role of excipient |
| 12 | Glycine | NIL | NIL | NIL |
| | Tri sodium citrate | 1.50 | 5.1 | Buffering agent |
| | dihydrate | | | |
| | Sodium dihydrogen | 0.87 | 5.6 | Buffering agent |
| | phosphate dihydrate | | | |
| | Sodium chloride | NIL | NIL | NIL |
| | Sucrose (2X) | 20.00 | 58.4 | Bulking Agent and |
| | | | | structure stabilizer |

| | |
|---|---|
| Fill Volume in mL | 0.50 |
| DS Concentration | 50 mg/mL |
| mg/mL | |

(J) Reconstitution buffer 12

| Excipient composition Reconstitution | Concentration |
|---|---|
| buffer 12 | mg/mL |
| Glycine | 5.62 |
| Tri sodium citrate dihydrate | 3.75 |
| Sodium dihydrogen phosphate dihydrate | 2.17 |
| Sodium chloride | 3.80 |
| Sucrose | NIL |
| Reconstitution Volume | 1.00 mL |

### Example 4: Optimization of Formulation Compositions for Lyophilisation and Reconstitution Buffer for Desirable Cake Attributes which is Qualitatively and Quantitatively Same as that of Ready-to-Use Liquid Formulation

In this approach excipients interfering the cake formation (higher sodium chloride content and higher buffer strength) were eliminated or reduced in the lyophilate and added to solution for reconstitution. The formulations tried with this approach are as follows:

TABLE 5
Composition obtained by removing or reducing the interfering
excipients and adding it to solution for reconstitution.
(A1) Formulation 13

| Formu- | | Concen- | Mo- | |
|---|---|---|---|---|
| lation | | tration | larity | |
| Number | Excipients | mg/mL | in mM | Role of excipient |
| 13 | Glycine | NIL | NIL | NIL |
| | Tri sodium citrate | 1.50 | 5.1 | Buffering agent |
| | dihydrate | | | |
| | Sodium dihydrogen | 0.87 | 5.6 | Buffering agent |
| | phosphate dihydrate | | | |
| | Sodium chloride | NIL | NIL | NIL |
| | Sucrose | 10.00 | 29.2 | Bulking Agent and |
| | | | | structure stabilizer |

| | |
|---|---|
| Fill Volume in mL | 1.00 |
| DS Concentration | 25 mg/mL |
| mg/mL | |

(A2) Reconstitution buffer 13

| Excipient composition Reconstitution | Concentration |
|---|---|
| buffer 13 | mg/mL |
| Glycine | 5.62 |
| Tri sodium citrate dihydrate | 3.00 |
| Sodium dihydrogen phosphate dihydrate | 1.73 |
| Sodium chloride | 3.80 |
| Sucrose | NIL |
| Reconstitution Volume | 1.00 mL |

(B1) Formulation 14

| Formu- | | Concen- | Mo- | |
|---|---|---|---|---|
| lation | | tration | larity | |
| Number | Excipients | mg/mL | in mM | Role of excipient |
| 14 | Glycine | 2.81 | 37.5 | NIL |
| | Tri sodium citrate | 1.50 | 5.1 | Buffering agent |
| | dihydrate | | | |
| | Sodium dihydrogen | 0.87 | 5.6 | Buffering agent |
| | phosphate dihydrate | | | |
| | Sodium chloride | NIL | NIL | NIL |
| | Sucrose | 10.00 | 29.2 | Bulking Agent and |
| | | | | structure stabilizer |

| | |
|---|---|
| Fill Volume in mL | 1.00 |
| DS Concentration | 25 mg/mL |
| mg/mL | |

(B2) Reconstitution buffer 14

| Excipient composition Reconstitution | |
|---|---|
| buffer 14 | mg/mL |
| Glycine | 2.81 |
| Tri sodium citrate dihydrate | 3.00 |
| Sodium dihydrogen phosphate dihydrate | 1.73 |
| Sodium chloride | 3.80 |
| Sucrose | NIL |
| Reconstitution Volume | 1.00 mL |

(C1) Formulation 15

| Formu- | | Concen- | Mo- | |
|---|---|---|---|---|
| lation | | tration | larity | |
| Number | Excipients | mg/mL | in mM | Role of excipient |
| 15 | Glycine | 5.62 | 75.0 | Bulking Agent in |
| | | | | Lyo |
| | Tri sodium citrate | 1.50 | 5.1 | Buffering agent |
| | dihydrate | | | |
| | Sodium dihydrogen | 0.87 | 5.6 | Buffering agent |
| | phosphate dihydrate | | | |
| | Sodium chloride | NIL | NIL | NIL |
| | Sucrose | 10.00 | 29.2 | Bulking Agent and |
| | | | | structure stabilizer |

| | |
|---|---|
| Fill Volume in mL | 1.00 |
| DS Concentration | 25 mg/mL |
| mg/mL | |

(C2) Reconstitution buffer 15

| Excipient composition Reconstitution | Concentration |
|---|---|
| buffer 15 | mg/mL |
| Glycine | NIL |
| Tri sodium citrate dihydrate | 3.00 |
| Sodium dihydrogen phosphate dihydrate | 1.73 |
| Sodium chloride | 3.80 |
| Sucrose | NIL |
| Reconstitution Volume | 1.00 mL |

(D1) Formulation 16:

| Formu- | | Concen- | Mo- | |
|---|---|---|---|---|
| lation | | tration | larity | |
| Number | Excipients | mg/mL | in mM | Role of excipient |
| 16 | Glycine | 5.62 | 75.0 | Bulking Agent in |
| | | | | Lyo |
| | Tri sodium citrate | 4.50 | 15.3 | Buffering agent |
| | dihydrate | | | |
| | Sodium dihydrogen | 2.60 | 16.6 | Buffering agent |
| | phosphate dihydrate | | | |
| | Sodium chloride | NIL | NIL | NIL |
| | Sucrose | 10.00 | 29.2 | Bulking Agent and |
| | | | | structure stabilizer |

| | |
|---|---|
| Fill Volume in mL | 1.00 |
| DS Concentration | 25 mg/mL |
| mg/mL | |

(D2) Reconstitution buffer 16

| Excipient composition Reconstitution | Concentration |
|---|---|
| buffer 16* | mg/mL |
| Glycine | NIL |
| Tri sodium citrate dihydrate | NIL |
| Sodium dihydrogen phosphate dihydrate | NIL |
| Sodium chloride | 3.80 |
| Sucrose | NIL |
| Reconstitution Volume | 1.00 mL |

*The solution for reconstitution may or may not contain 0.9% benzyl alcohol as preservative.

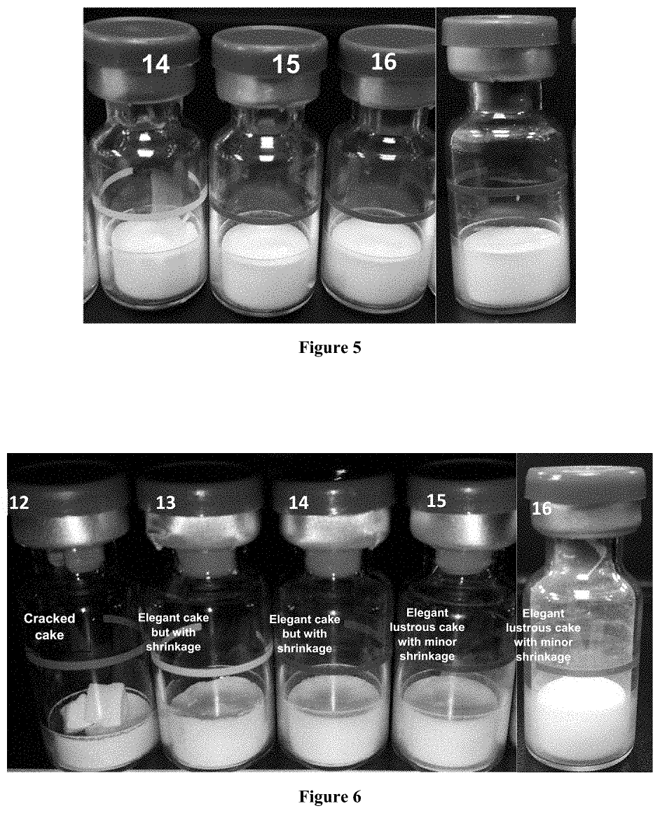

The results of above formulations are depicted in FIG. 5 and are as follows:

Formulation No. 13: Elegant cake with shrinkage
Formulation No. 14: Elegant cake with shrinkage
Formulation No. 15: Elegant lustrous cake with minor shrinkage
Formulation No. 16: Elegant lustrous cake with minor shrinkage

The formulations form the final cycle (Formulation number 13, 14, 15 and 16) were charged on stability at 40° C. for 2 weeks to evaluate further shrinkage or melt back due to moisture content.

The results of above formulations for stability at 40° C. after 2 weeks are depicted in FIG. 6 and are as follows:

Formulation No. 12: Cracked cake
Formulation No. 13: Elegant cake but with shrinkage
Formulation No. 14: Elegant cake but with shrinkage
Formulation No. 15: Elegant lustrous cake with minor shrinkage
Formulation No. 16: Elegant lustrous cake with minor shrinkage

The reconstitution time for formulation number 12, 13, 14, 15 and 16 is summarized in table below:

TABLE 6
Reconstitution time for various formulations
evaluated for cake formation.

| | Average Reconstitution |
|---|---|
| Formulation No. | time in min |
| 12 | 1.31 |
| 13 | 1.10 |
| 14 | 1.00 |
| 15 | 1.23 |
| 16 | 1.00 |

The reconstitution time of all the formulation was less than 2 minutes.

On the basis of stability results at 40° C. for two weeks it was found that formulation no. 13, 14, 15 and 16 formed a stable cake and had no major changes. By increasing the amount of glycine to 2 fold, the cake of formulation no. 15 and 16 was comparable to the marketed formulation (Enbrel®). The formulation no. 12 formed an elegant but brittle cake which cracked during handling.

The moisture content of formulation no. 15 and 16 was determined by reconstituting the Hydranal solution in the etanercept lyophilized drug product vial followed by centrifugation and injecting the supernatant in Karl fisher for moisture content estimation. The moisture content values were found to be <2% w/w. The data for moisture content is as given below.

TABLE 7
Moisture content of formulation no. 15 and 16
in lyophilization cycle trial 14, 15 and 16.

| | | Targeted average* | Average* |
|---|---|---|---|
| | | moisture content | Moisture content |
| Formulation | Trial No. | % w/w | % w/w |
| 15 | Trial 14 | <2% | 1.10% |
| 15 | Trial 15 | | 1.30% |
| 16 | Trial 16 | | 1.10% |

Note:
*Average of 10 vials.

### Example 5: Optimization of Lyophilization Cycle

The lyophilization cycles used for development of these formulations involved freezing of drug substance from −40° C. to −50° C. at a rate of −0.6° C./min to 1.5° C./min followed by and annealing at −25° C. to −30° C. at a rate of 0.1° C./min to 0.3° C./min. Slow freezing results in formation of larger ice crystals and interstitial pores and in turn increases mass/moisture transfer during drying process. Faster rate of drying forms smaller ice crystals and interstitial pores in turn reducing the rate of moisture transfer during drying process and increasing moisture content in the lyophilized cake. Primary and secondary drying involved stepwise increment of temperatures and holding at each step for 4 to 6 hours. The pressure in the lyophilization chamber was maintained at 100 to 50 mTorr during primary and secondary drying. The total lyophilization cycle varied from 48 to 72 hours during development stage. The final cycle was approximately of 48 hours.

### Example 6: Comparison Between Formulated Bulk Solution of Formulation 15 and 16

The formulated bulk for formulation 15 and 16 were analyzed for its purity after buffer exchange step and table 8 below summarizes the results obtained from SE-HPLC analysis.

TABLE 8
Comparison between purity of formulated bulk
for formulation 15 and 16 by SE-HPLC.

| | Formulation 15 | | | Formulation 16 | | |
|---|---|---|---|---|---|---|
| | % High | | % Low | % High | | % Low |
| | Molecular | | Molecular | Molecular | | Molecular |
| SAMPLE STAGE/ | Weight | % | Weight | Weight | % | Weight |
| DESCRIPTION | impurities | Monomer | impurities | impurities | Monomer | impurities |
| Control DS | 3.2 | 96.4 | 0.3 | 3.2 | 96.4 | 0.3 |
| (Before Buffer | | | | | | |
| exchange) | | | | | | |
| After Buffer | 3.4 | 95.5 | 1.1 | 3.3 | 96.4 | 0.4 |
| exchange step | | | | | | |
| Before | 2.7* | 94.4* | 2.8* | 3.3* | 96.1* | 0.6* |
| Lyophilization | | | | | | |

NOTE:
*Purity values after ~1 week storage at 2° C. to 8° C.

From the above data it can be observed that the low molecular weight impurities were increased in formulation 15 while the purity values remained unchanged for formulation 16 when analysed immediately after buffer exchanging step. Formulation 15 analysed after 1 week's storage at 2° C. to 8° C. shows further increase in low molecular weight impurities. Formulation 16 analysed after 1 week's storage at 2° C. to 8° C., shows relatively less degradation. This shows that reduction in tri-sodium citrate content would increase in low molecular weight impurities.

### Example 7: Comparison of Tm (Melting Point) Values of Formulation 15 and 16 by DSC (Differential Scanning Colorimetry)

Three thermal transition temperatures of Tm₁, Tm₂, and Tm₃ for etanercept were observed. These indicated unfolding states at an extra-cellular ligand binding portion, tumour necrosis factor receptor (TNFR; Tm1) and the presence of two domains originating from the Fc component of Etanercept containing the domains of CH2 (Tm2), CH3 (Tm3). The first thermal transition is usually taken into account for stability determination because it occurs at lower temperature than the one for the CH3 domain.

The comparison of Tₘ values of formulation number 15 and formulation number 16 are summarized in table 9 below.

TABLE 9
Melting temperatures (Tm) of formulation 15 and 16 by DSC.

| Formulation | | | |
|---|---|---|---|
| number | Tm₁ | Tm₂ | Tm₃ |
| Control DS | 58.26 | 70.21 | 81.94 |
| 15 | 56.52 | 69.33 | 85.45 |
| 16 | 57.56 | 69.96 | 82.03 |

Form the above data it can be observed that the Tₘ values of formulation number 15 are significantly lower than the Control DS. However, formulation 16 had Tₘ values higher than formulation 15 and were close to the Control DS, suggesting improved stability. Tm1 representing unfolding of the TNFR receptor and Tm2 representing unfolding CH2 domain are considered key indicators of stability by nano-DSC analysis.

Based on above results, the most preferable formulation was thus formulation no. 16 and was finalized for further stability batches which is qualitatively as well as quantitatively same as that of an already established ready-to-use liquid formulation as mentioned in Table 2.

### Example 8: Stability of Formulation 16

Three batches (Batch 1, 2 and 3) of formulation number 16 were charged on development stability at real time (2° C. to 8° C. up to 6 months), accelerated (25° C. up to 6 months) and stressed conditions (40° C. up to 1 month) and were analysed for their purity by SEHPLC and HI HPLC. Formulation 16 was prepared and lyophilized according to lyophilization cycle described in Example 5. The lyophilized formulation was incubated at real time (2° C. to 8° C.), accelerated (25° C.) and stressed conditions (40° C.). The lyophilized formulation at different time point (i.e. after 1 month for stressed conditions and after 6 months for real time and accelerated conditions) was reconstituted with its corresponding reconstitution buffer 16 and was analysed for its purity by SE-HPLC and HI-HPLC.

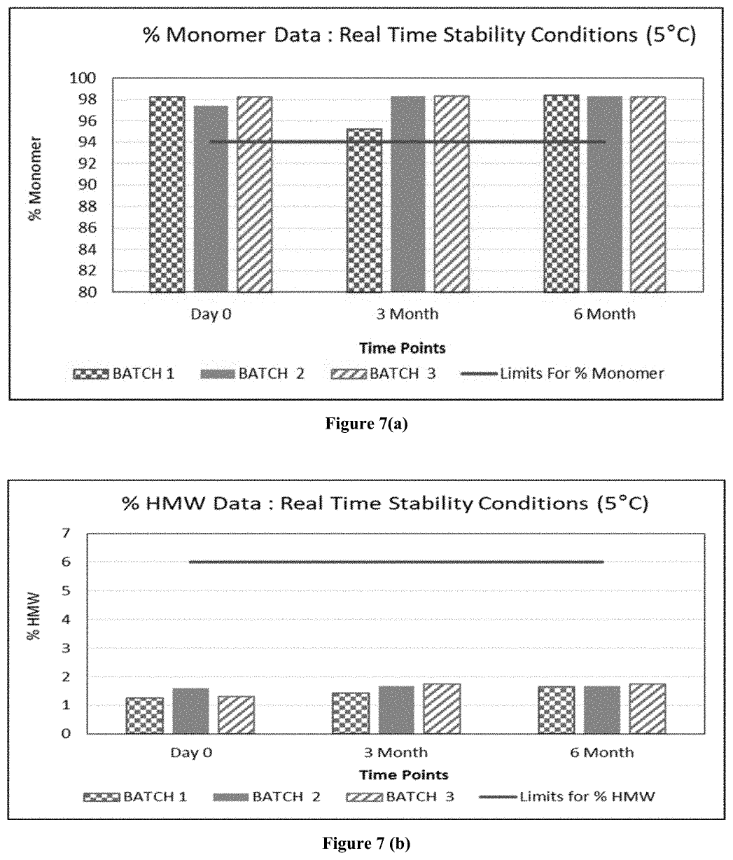

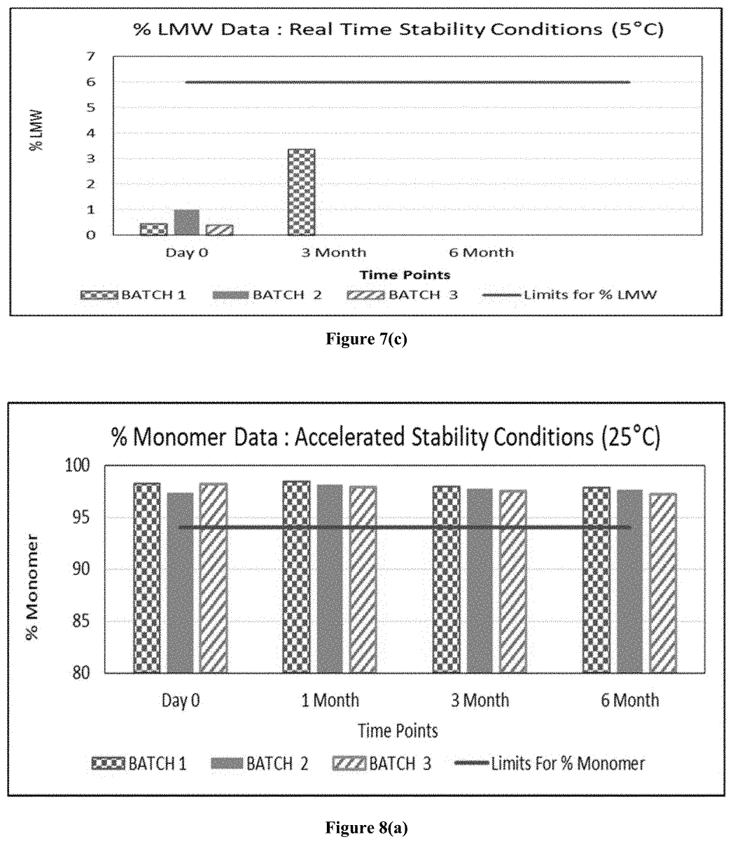

From the FIG. 7, it can be observed that, although within specifications, % BMW show a slight increase over a period of time. The abrupt increase in % LMW can be attributed to analytical variation, as no such change was observed at accelerated or stressed conditions and further time point (i.e. 6 months) at real time conditions. Rest all the quality attributes (% monomer content and % LMW) analysed by SE-HPLC are within the define specifications and had no impact of storage at 2° C. to 8° C. up to 6 months. Thus, it can be concluded that formulation 16 is stable at real time conditions (2° C. to 8° C.) up to 6 months time point.

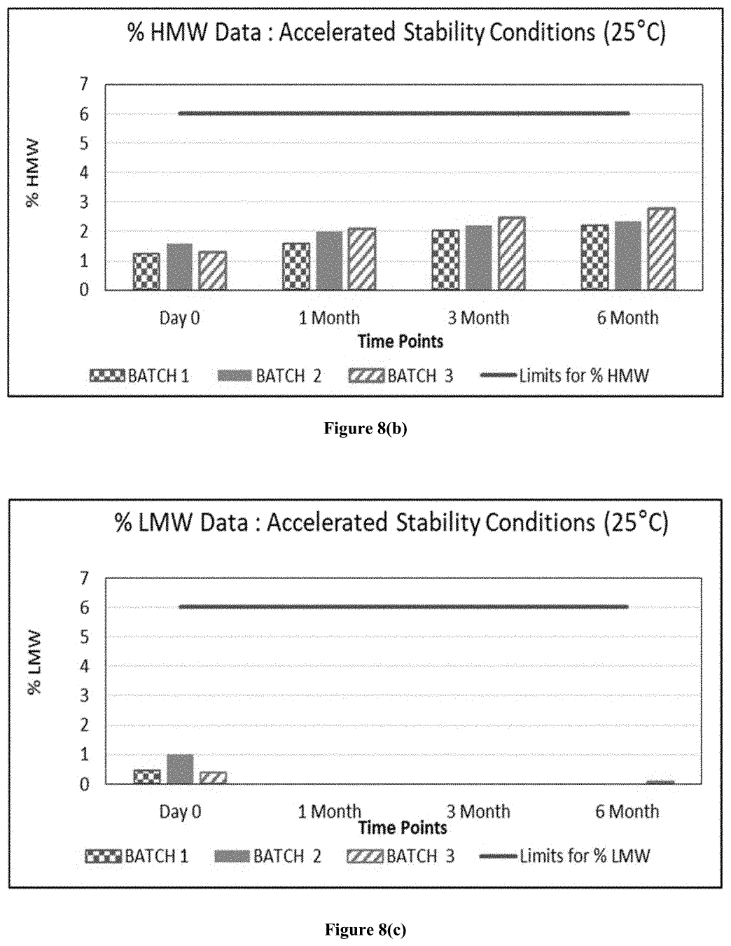

From the FIG. 8, it can be observed that, % HMW show a gradual increase up to 6 months time. However, the increase in % BMW was up to 2 to 3%, which is well within the specifications. Rest all the quality attributes (% monomer content and % LMW) analysed by SE-HPLC are within the defined specifications and had no impact of storage at 25° C./60% RH up to 6 months time point. Thus, it can be concluded that formulation 16 is stable up to 6 months time point at accelerated conditions (25° C./60% RH).

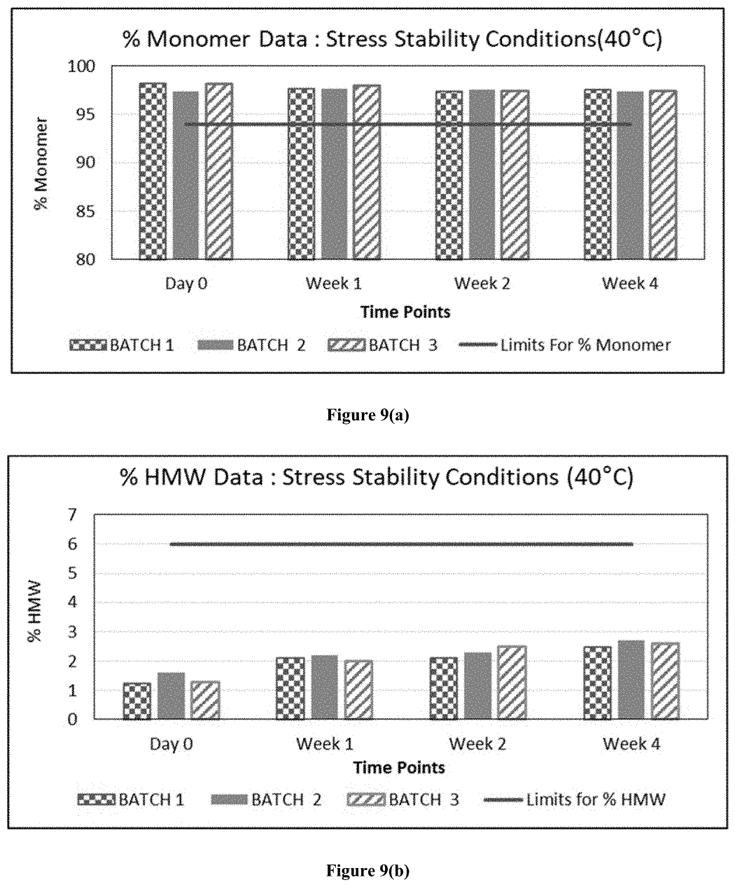

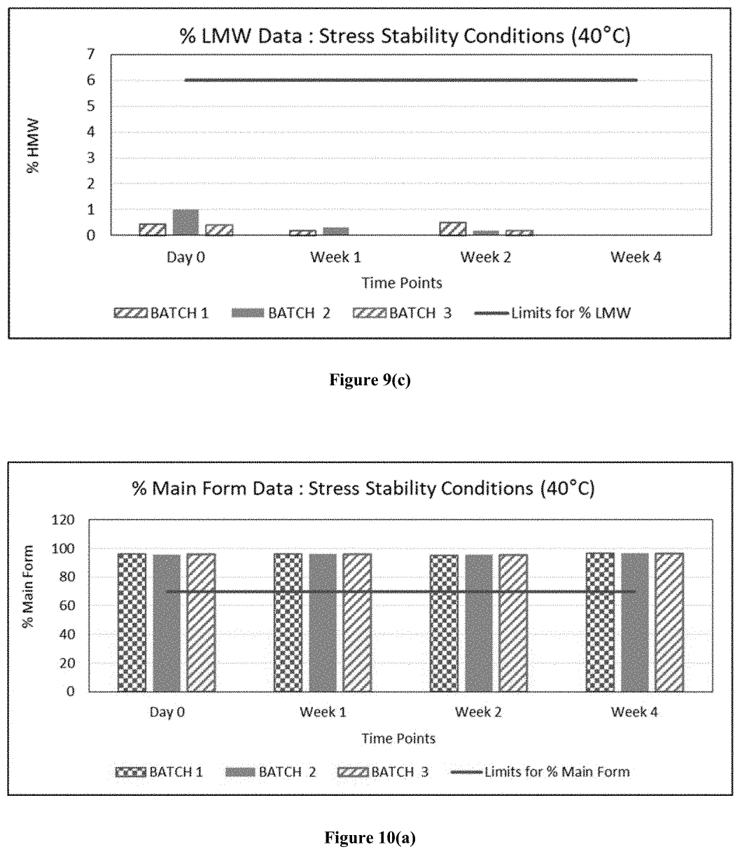

The stress stability data was in agreement with the accelerated stability data. From the FIG. 9, it can be observed that, similar to accelerated stability data, % HMW show a gradual increase up to 1 month time up to 2 to 3%, which is within the specifications. The remaining quality attributes (% monomer content and % LMW) analysed by SE-HPLC were within the defined specifications and had no impact of storage at 40° C./75% RH up to 1 month time point. Thus, from the above SE-HPLC data it can be concluded that formulation 16 is stable up to 1M time point at stress conditions (40° C./75% RH).

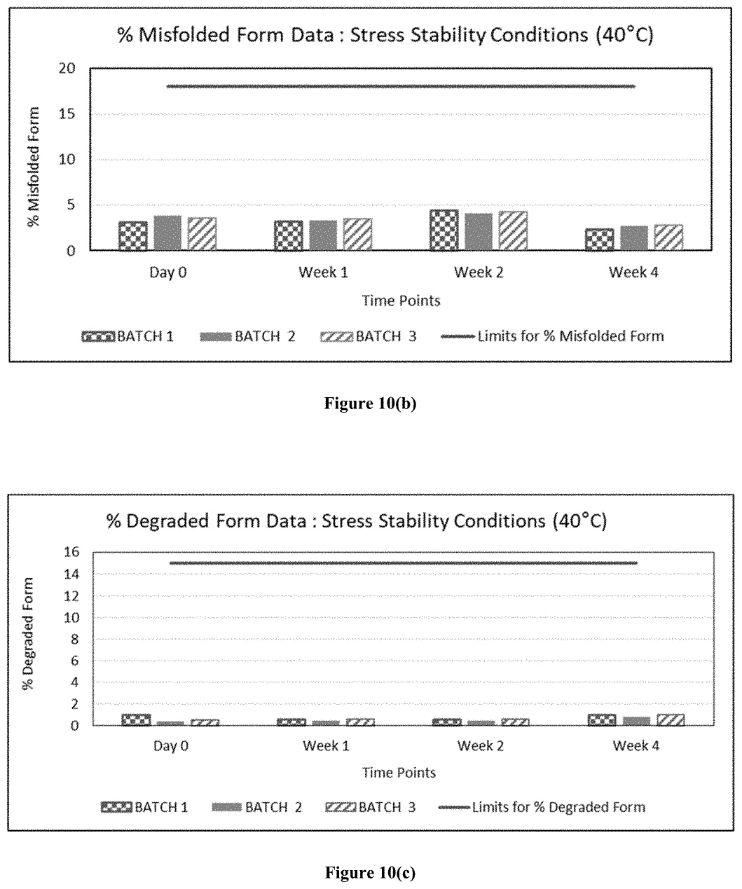

FIG. 10 summarizes the stability data by HI HPLC for formulation 16 at stressed conditions 40° C./75% RH up to 1 month.

Form the stability data at 40° C./75% RH, it can be observed that formulation number 16 did not have any impact on the purity by HI HPLC up to one month's time.

Thus, purity by HI HPLC for formulation number 16 was not impacted under stressed conditions. Similar observations were seen at real time and accelerated conditions, suggesting that, when formulation number 16 is stored at real time or accelerated conditions, there was no impact on purity by HI HPLC.

Apart from the impact on product quality following table summarizes information on moisture content and pH post reconstitution, at real time condition (2° C. to 8° C.).

TABLE 10
% Moisture content of formulation 16 at
real time conditions (2° C. to 8° C.).
% Moisture Content:
Real Time Stability (5° C.)

| Batch Label | Day 0 | 6 Months |
|---|---|---|
| Batch 1 | 1.4 | 1.5 |
| Batch 3 | 1.3 | 1.6 |
| Batch 4 | 1.1 | 1.5 |

TABLE 11
pH formulation 16 at real time conditions (2° C. to 8° C.) up to 3 months.
Real Time Stability (5° C.)

pH

| Batch Label | Day 0 | 3 Months | Acceptance Criteria |
|---|---|---|---|
| Batch 1 | 6.23 | 6.24 | pH = 6.3 ± 0.2 |
| Batch 3 | 6.23 | 6.22 | post reconstitution |
| Batch 4 | 6.20 | 6.21 | |

From the above data it was observed that, the final moisture content for formulation 16 was <2% even after storage at real time conditions up to 6 months and pH post reconstitution with reconstitution buffer 16 was within the acceptance criteria of 6.3±0.2 at real time conditions up to 3 months.

From the above data and examples it can be concluded that a stable lyophilized dosage form of protein, which upon reconstitution has the same composition as that of an already established or developed ready-to-use liquid dosage form, comprising attributes such as 1) elegant & stable cake which is free from collapse or melt back 2) minimum reconstitution time 3) minimum particulates upon reconstitution, can be manufactured by reducing/removing excipients interfering with cake formation and adding the same to solution for reconstitution. The formulations of the invention have several benefits relative to other protein lyophilized composition, including reduced cost of development, minimum reconstitution time, minimum particulates upon reconstitution and a relatively stable elegant commercial viable protein lyophilized formulation. Above examples demonstrate that a ready-to-use liquid formulation which does not form cake as such can be modified as described here resulting in elegant cake. This study thus exemplifies lyophilization strategy to convert an established or developed ready-to-use solution liquid dosage form of protein to lyophilized dosage form of the same protein which upon reconstitution has the same composition as that of the ready-to-use liquid formulation.

1. A stable lyophilized dosage form of protein comprising a lyophilate and a reconstitution liquid wherein the lyophilized dosage form upon re-constitution with the reconstitution liquid is qualitatively as well as quantitatively same as that of ready-to-use liquid dosage form of the same protein.

2. The stable lyophilized dosage form of protein according to claim 1, wherein said protein comprises an antibody, an antibody fragment, a fusion protein or a peptide.

3. The stable lyophilized dosage form of protein according to claim 1, wherein said protein is etanercept.

4. The stable lyophilized dosage form of protein according to claim 1, wherein said lyophilized dosage form of protein is stable at real time and accelerated stress stability conditions for at least six months and at stress stability conditions for at least one month.

5. A stable lyophilized dosage form of protein comprising a lyophilate and a reconstitution liquid wherein the lyophilate has reduced concentration of salt.

6. The stable lyophilized dosage form of protein according to claim 5, wherein the said lyophilate has reduced concentration of salt as well as buffer.

7. The stable lyophilized dosage form of protein according to claim 5, wherein the said lyophilate has salt at a concentration of less than or equal to 150 mM and buffer at a concentration of less than or equal to 32 mM.

8. The stable lyophilized dosage form of protein according to claim 5, wherein said protein comprises an antibody, an antibody fragment, a fusion protein or a peptide.

9. The stable lyophilized dosage form of protein according to claim 5, wherein said protein is etanercept.

10. The stable lyophilized dosage form of protein according to claim 5, wherein said lyophilized dosage form of protein is stable at real time and accelerated stress stability conditions for at least six months and at stress stability conditions for at least one month.

11. A stable lyophilized dosage form of protein comprising a lyophilate with salt at a concentration of less than or equal to 150 mM along with buffer at a concentration of less than or equal to 32 mM respectively and a reconstitution solution comprising salts and optionally optionally buffer, bulking agent, solvent, preservative or a combination thereof, wherein the said lyophilized dosage form is qualitatively as well as quantitatively same as that of ready-to-use liquid dosage form of the same protein.

12. The stable lyophilized dosage form of protein according to claim 10, wherein said protein comprises an antibody, an antibody fragment, a fusion protein or a peptide.

13. The stable lyophilized dosage form of protein according to claim 10, wherein said protein is etanercept.

14. The stable lyophilized dosage form of protein according to claim 10, wherein said lyophilized dosage form of protein is stable at real time and accelerated stress stability conditions for at least six months and at stress stability conditions for at least one month.
